(19) United States
(12) Patent Application Publication
(10) Pub. No.: US 2007/0264324 A1
(43) Pub. Date: Nov. 15, 2007

(54) Use of Vegetable Anthraquinone Derivatives and Vegetable Polysaccharides For Treating Human Immunodeficiency Virus (HIV)

(76) Inventors: Zhanqiu Yang, Shenzhen (CN); Hong Xiao, Shenzhen (CN); Shouhui Zhu, Shenzhen (CN); Xiang Peng, Shenzhen (CN)

Correspondence Address: Zhanqiu YANG; Hong XIAO;;Shouhui ZHU; Xilang PENG, No. 2, Road of Lanshan, High-Tech District, Guandpng 51900 (CN), Shenzhen, 51800, CN

(21) Appl. No.: 11/749,867

(22) Filed: May 17, 2007

(30) Foreign Application Priority Data
Nov. 14, 2005 (CN) PCT/CN05/01919
Nov. 18, 2004 (CN) 200410052355.1

(51) Int. Cl. A61K 31/715; A61K 31/12; A61K 9/48; A61K 9/20

(52) U.S. Cl. 424/451; 424/464; 424/779; 514/54; 514/680

(57) ABSTRACT

The use of vegetable anthraquinone derivatives and vegetable polysaccharides for treating human immunodeficiency virus(HIV); the anthraquinone derivatives and polysaccharides were obtained by CO₂ supercritical extraction from plants, then the vegetable anthraquinone derivatives and vegetable polysaccharides alone or the mixture thereof were subject to test for inhibiting HIV virus, HIV virus was added to the culture plate containing emodins and Rheum hotaoense polysaccharides good growth of cells and reduction of virus amount were observed. So said emodins and Rheum hotaoense polysaccharides can be used as active ingredient to preparing drugs or foods for treating AIDS.

### CROSS-REFERENCE TO RELATED APPLICATIONS

This application claims the benefit of International Application No. PCT/CN2005.001919 filed on Nov. 14, 2005, which in turn claims the benefit of China Application No. 200410052355.1 filed on Nov. 18, 2004.

### BACKGROUND OF THE INVENTION

1. Field of Invention

The present invention relates to the use of emodin and Rheum rhaponticum polysaccharide (RRP), and more particularly, to the innovative antiviral application of emodin and RRP against human immunodeficiency virus (HIV).

2. Description of Related Art

Since its first recognized case in 1981, AIDS has become a major world pandemic. In China, the number of people infected with HIV is 840,000, with 80,000 people have AIDS and 160,000 deaths attributed to the disease. Researchers around the world are seeking cures for HIV or AIDS, with no success in finding a drug with no side effects while still being effective.

There are currently three ways for treating AIDS: the first targets AIDS itself, the second targets the immune system, while the third targets opportunistic infections. More specifically, the first deals with the cause of the disease (HIV); the second encompasses complementary therapies enhancing the immune system of the patient, and the third avoids the exposure to other infectious agents. Anti-HIV chemical agents (such as Zidovudine) are shown to be very effective, albeit with severe side effects, intolerance on the side of patients and drug-resistance developed by HIV, leading to return of symptoms once treatment is stopped, all of which are yet to be resolved. The latter two ways are merely complementary and prophylactic approaches, bearing no effects in treating patients developing AIDS. Therefore, it is imperative to find an effective HIV inhibiting agent with little or no side effects. Traditional Chinese herbal medicine shows great potential in this aspect. According to publications researched, herbal medicine with anti-HIV and anti-AIDS effects are yet to be announced. There are reports on anti-HIV effects exhibited by glycyrrhizin and Rheum rhaponticum, with no insightful research on the former and no specific anti-HIV substance discovered for the latter, which deems these reports as directing precursors at best.

### BRIEF SUMMARY OF THE INVENTION

The present invention relates to the innovative antiviral application of emodin and Rheum rhaponticum polysaccharide (RRP) against human immunodeficiency virus (HIV). One object of the present invention is to provide a superior antiviral agent for incorporating in the treatment of AIDS. Other objects will become apparent from a consideration of the ensuing description.

### DETAILED DESCRIPTION OF THE INVENTION

Disclosure of the Invention

The present invention relates to the innovative antiviral application of emodin and Rheum rhaponticum polysaccharide (RRP) against human immunodeficiency virus (HIV), providing a superior antiviral agent for incorporating in the treatment of AIDS.

The technical solution provided by the present invention includes:
1. the antiviral application of emodin and RRP against HIV; and
2. a test, comprising:
   i. employing supercritical CO₂ fluid extraction method to extract emodin and RRP from Rheum rhaponticum herbs, and
   ii. testing the HIV-inhibiting effectiveness of emodin and RRP respectively and as a mixture thereof.

The present invention relates to using emodin and RRP to inhibit HIV in an exogenous (however endocellular) environment, in order to attest their anti-HIV effectiveness. More specifically, the invention includes:
1. Preparing emodin and RRP solutions with various concentrations (70-98% for emodin, and 80-95% for RRP).
2. Culturing test subjects containing 100 μL of cells using cell incubator under 37° C. and 5% CO₂ for 24 hours, forming monolayer on each culture media.
3. Testing the toxicity of the agents: apply Rheum rhaponticum extractions to the monolayer on culture medium. Maintain the culture media under 37° C. and 5% CO₂ for 72 hours, and observe the monolayer for cellular viability. Repeat the test 4 times for every concentration of emodin and RRP solution/mixture and choose the concentrations that do not affect the growth of cells as the standard concentrations for further tests. These standard concentrations of emodin range between 70% and 98%, and those of RRP range between 80% and 95%.
4. Testing the anti-HIV effectiveness of emodin and RRP: apply a specific concentration of HIV to those culture medium containing viable cells and already applied with emodin and/or RRP. Observe the viability of cells, and compare them with those in the control groups.

The present invention is characterized by:

The test:
(1) Control group: apply HIV to culture medium with no emodin or RRP.
(2) Test group: A. apply HIV to culture medium with mixtures of emodin and RRP; B. apply HIV to culture medium containing viable cells and already applied with emodin; C. apply HIV to culture medium containing viable cells and already applied with RRP.
(3) Control group: A. apply HIV to culture medium without mixtures of emodin and RRP; B. apply HIV to culture medium containing viable cells but no emodin; C. apply HIV to culture medium containing viable cells but no RRP.

Results: the cells in the control group are destroyed, and the group sees an increased virus load; while test group A (applied with mixtures of emodin and RRP) exhibits a good cell viability and decreased virus load. These results show that substance used in test group A has a good inhibiting effect to HIV. The test is repeated 4 times, all of which have the same results. Therefore the above mentioned emodin and RRP extraction from Rheum rhaponticum herbs may be employed as the active ingredient through the following means for the treatment of AIDS: capsules, tablets, granules and injections.

### DETAILED DESCRIPTION OF THE EMBODIMENT

1. Test subject: TC740 cells. Cultured using RPMI-1640 medium added with bovine serum (10% concentration), 100U/mL of penicillin, 100 μg/mL of streptonigrin, and general cell culture agents.
Virus: 10⁴⁻⁸ CID50/0.1 mL HIV, proliferated using TC 740 cells, with more than 75% showing cytopathic effects.
emodin and RRP: extracted from Rheum rhaponticum herbs using supercritical CO₂ fluid extraction method.

2. Cell culture method: apply 100 μL TC740 cell with a concentration of 2×10³⁻⁵/mL to culture medium, and maintain the medium under 37° C. and 50% CO₂ between 12 and 24 hours.

3. Cytotoxicity test: apply an emodin/RRP mixture with a concentration between 1600 μg/mL and 25 μg/mL to the culture medium, and maintain the medium under 50% CO₂ for 72 hours before observing.

4. The test:
(1) Control group: apply HIV to culture medium with no emodin or RRP.
(2) Test group: apply HIV to culture medium with the emodin/RRP mixture.
(3) Results: the cells in the control group are destroyed, and the group has an increased virus load; while the test group exhibits a good cell viability and decreased virus load. The test is repeated 4 times, all of which have the same results.

Therefore the above mentioned emodin and RRP extraction from Rheum rhaponticum herbs may be employed as the active ingredient though the following means for the treatment of AIDS: capsules, tablets, granules and injections.

Aside from the above mentioned extraction method using Rheum rhaponticum herbs, emodin and RRP may also be extracted from other emodin or RRP containing herbs/medicine. Thus, all AIDS treatments employing emodin and/or RRP are within the scope of the present invention.

What is claimed is:

1. An antiviral application of emodin and Rheum rhaponticum polysaccharide against human immunodeficiency virus.

2. The antiviral application of emodin and Rheum rhaponticum polysaccharide against human immunodeficiency virus of claim 1, characterized in that emodin and Rheum rhaponticum polysaccharide are used as the active ingredients in the preparation of capsules, tablets, granules and injections.
